(19) United States
(12) Patent Application Publication
(10) Pub. No.: US 2017/0172534 A1
(43) Pub. Date: Jun. 22, 2017

(54) THORACIC IMAGING FOR CONE BEAM COMPUTED TOMOGRAPHY

(71) Applicant: THE UNIVERSITY OF SYDNEY, Sydney, New South Wales (AU)

(72) Inventors: Chun-Chien SHIEH, Arncliffe, New South Wales (AU); John KIPRITIDIS, Rhodes, New South Wales (AU); Ricky O'BRIEN, Rozelle, New South Wales (AU); Zdenka KUNCIC, Sydney, New South Wales (AU); Paul KEALL, Greenwich, New South Wales (AU)

(73) Assignee: THE UNIVERSITY OF SYDNEY, Sydney, New South Wales (AU)

(21) Appl. No.: 15/325,357

(22) PCT Filed: Jul. 23, 2015
(86) PCT No.: PCT/AU2015/000434

§ 371 Date: Jan. 10, 2017

(30) Foreign Application Priority Data
Jul. 23, 2014 (AU) 2014902846

(51) Int. Cl. A61B 6/00; G06T 7/11; A61B 5/00; G06T 11/00; A61B 6/03; A61B 5/055

(52) U.S. Cl. CPC A61B 6/5258; A61B 6/032; A61B 6/4085; A61B 5/055; A61B 5/7203; A61B 6/505; A61B 6/5205; A61B 6/5217; G06T 11/008; G06T 7/11; G06T 2207/10081; G06T 2207/10088

(57) ABSTRACT

A method of adaptive suppression of over-smoothing in noise/artefact reduction techniques such as total variation minimization or other compressed sensing strategies for Cone Beam Computed Tomography (CBCT) images, the method including the steps of: (a) inputting a CBCT image; (b) identifying the anatomical structures of interest in the CBCT images by exploiting their likely shapes, attenuation coefficients, sizes, positions, or any other similar features that can be used to identify them from an image; (c) extracting intensity, gradient, or other image-related information of the identified anatomical structures from the CBCT image; (d) adaptively suppressing over-smoothing in noise/artefact reduction techniques such as total-variation minimization or other compressed sensing strategies at the anatomical structures of interest using the information of the anatomical structures extracted previously.

### FIELD OF THE INVENTION

The present invention relates to the field of adaptive image processing of noisy imagery, such as those generated by Cone Beam Computed Tomography (CBCT) and analogous image generation techniques, and, in particular discloses a more effective means of processing of adaptive smoothing CBCT images.

### BACKGROUND

Any discussion of the background art throughout the specification should in no way be considered as an admission that such art is widely known or forms part of common general knowledge in the field.

The present description includes a number of references to external publications, indicated within brackets. Those references appear hereinafter in the description.

In image-guided radiation therapy (IGRT), the linear accelerator (linac)-mounted cone-beam computed tomography (CBCT) imaging unit allows the tumor position to be verified immediately prior to treatment. However, conventional three-dimensional (3D) CBCT suffers from motion blur in the thoracic region due to respiratory motion. Four dimensional (4D) CBCT is an emerging imaging technique used to resolve tumor motion. In 4D CBCT, projection images are sorted into phase-correlated subsets (or “phase bins”) corresponding to different respiratory phases, from which temporally resolved images are reconstructed (Sonke et al, 2005). The use of 4D CBCT improves both target coverage and normal tissue avoidance in thoracic IGRT (Harsolia et al, 2008).

The reconstruction of high quality 4D CBCT images is difficult because of the sparse angular sampling caused by projection allocation. In current practice, projection images in each phase bin are reconstructed into a 3D volume using the Feldkamp-Davis-Kress (FDK) algorithm (Feldkamp et al, 1984), which is essentially an approximate filtered backprojection. Despite its computational efficiency, FDK produces severe noise and streaking artifacts in 4D CBCT images due to projection under-sampling. The Mckinnon-Bates algorithm (Mckinnon and Bates, 1981; Leng et al, 2008a; Zheng et al, 2011) reduces noise and streaking by exploiting the motion blurred yet high signal-to-noise ratio (SNR) 3D CBCT image. However, the overall improvement in image quality is limited, and residual motion artifacts remain an issue (Bergner et al, 2010).

The emergence of compressed sensing (CS) theory (Candes et al, 2006; Donoho, 2006) enables iterative reconstruction of under-sampled datasets via minimizing the 11-norm of suitable “sparsifying transforms” of the images. For applications in CBCT, minimization of the 11-norm of the gradient image, i.e. total-variation (TV), has been shown to be efficient for noise and streaking reduction (Sidky and Pan, 2008; Choi et al, 2010; Ritschl et al, 2011). A commonly used framework for iterative TV minimization reconstruction is the adaptive-steepest-descent projection-onto-convex-sets (ASD-POCS) algorithm (Sidky and Pan, 2008), which consists of iterative alternations between a projection-onto-convex-sets (POCS) component to enforce the data fidelity constraint and a TV minimization component to reduce noise/streaking. Although TV minimization reconstruction results in much less noise and streaking artifacts compared to FDK and MKB, it is prone to over-smoothing fine anatomical structures as the TV minimization component tends to reduce intensity variations due to both noise/streaking and anatomical structures indistinguishably. In addition, TV minimization reconstruction often converges slowly, making it computationally inefficient and unfeasible for clinical use (Bergner et al, 2010).

By incorporating certain prior knowledge of the volume of interest into a CS based algorithm, a noise- and streak-reduced yet sharper solution image and a faster convergence can be achieved. In the prior image constrained compressed sensing (PICCS) algorithm (Chen et al, 2008), prior knowledge is incorporated by imposing similarity between the solution image and a high SNR prior image. This additional constraint accelerates the convergence towards a solution image that shares similar high SNR traits with the prior image. In 4D CBCT, the motion blurred 3D CBCT image and the MKB image are suitable prior image choices (Leng et al, 2008b), as both are reasonable estimates of the reconstructed volume, and are higher in SNR than the FDK image. However, as the solution is often biased towards the prior image due to the stiff similarity constraint, the reconstruction may suffer from migration of residual motion artifacts and noise/streaking from the prior image (Bergner et al, 2010).

Another type of prior strategy involves the minimization of spatially adaptive TV. By applying a spatial weighting based on the gradient information of the image to the TV calculation, TV minimization can be suppressed adaptively at certain regions/pixels to preserve edges and structures. The gradient information exploited, such as the magnitude of the image gradient (Strong et al, 1997) or the difference curvature (Chen et al, 2010), can be viewed as the prior knowledge for edge detection. These strategies are widely applied in image restoration (Chantas et al, 2010; Dong et al, 2013; Yuan et al, 2013), and have also been demonstrated for low-dose CT reconstructions (Tian et al, 2011; Liu et al, 2012). However, gradient based edge detection is not robust to conspicuous artifacts and spatially inhomogeneous noise, both of which are commonly seen in 4D CBCT.

### SUMMARY OF THE INVENTION

It is an object of the invention, in its preferred form to provide an improved form of processing of images including CBCT images.

In accordance with a first aspect of the present invention, there is provided a method of adaptive suppression of over-smoothing in noise/artefact reduction techniques such as total variation minimization or other compressed-sensing strategies for Cone Beam Computed Tomography (CBCT) images, the method including the steps of: (a) inputting a CBCT image; (b) identifying the anatomical structures of interest in the CBCT images by exploiting their likely shapes, attenuation coefficients, sizes, positions, or any other similar features that can be used to identify them from an image; (c) extracting intensity, gradient, or other image-related information of the identified anatomical structures from the CBCT image; (d) adaptively suppressing over-smoothing in noise/artefact reduction techniques such as total-variation minimization or other compressed sensing strategies at the anatomical structures of interest using the information of the anatomical structures extracted previously.

In accordance with a further aspect of the present invention, there is provided a method of adaptive suppression of over-smoothing in noise/artefact reduction techniques such compressed-sensing strategies for anatomy imaging modalities, the method including the steps of: (a) inputting an imaging modality image; (b) identifying at least one anatomical structure of interest in the imaging modality image; (c) extracting intensity, gradient, or other image-related information of the identified anatomical structures from the imaging modality image; and (d) adaptively suppressing over-smoothing in noise/artefact reduction at the anatomical structures of interest using the information of the anatomical structures extracted previously.

In some embodiments, the anatomical structures of interest can include at least one of a soft tissue region, lung or airway region, bony anatomy, pulmonary region, or other similar anatomical structures/regions.

The preferred embodiment provides a method to adaptively suppress over-smoothing of anatomical structures in noise/artefact reduction techniques such as TV minimization or other compressed sensing image enhancement strategies, the preferred embodiment provides for the approximate identification of anatomical structures of interest from an image and uses them as a prior. The thoracic region consists of several distinct anatomical structures of interest in a CT or CBCT image: soft tissue, lungs/airways, bony anatomy, pulmonary details (tumors, vessels, and bronchus walls inside the lungs), and other similar anatomical structures/regions. These structures can be identified based on the general knowledge of the thoracic anatomy, e.g. the likely attenuation coefficients, positions, and shapes of each structure. By exploiting general anatomical knowledge, anatomical structures can be automatically segmented via strategies such as intensity thresholding, connectivity analysis, region growing, and morphological operators (Haas et al, 2008; van Rikxoort et al, 2009; Volpi et al, 2009; Vandemeulebroucke et al, 2012).

In some embodiments, the method can be applied to different imaging modalities other than CBCT, such as Computed Tomography (CT) and Magnetic Resonance Images (MRI).

In some embodiments, the method can be applied to different anatomical sites other than the thoracic region, such as the head and neck region or the prostate. In the case of a different anatomical site other than the thoracic region, the anatomies to be identified and used as a guidance for suppressing over-smoothing are replaced by the major anatomical structures in the corresponding anatomical site.

In accordance with a further embodiment of the present invention, there is provided a method of improving CBCT image reconstruction of a first body having anatomical structures, the method including the steps of: (a) determining a segmented anatomy prior for the first body delineating the anatomical structures; and (b) utilizing the segmented anatomy prior to modulate a total variation minimization of the CBCT image.

In some embodiments, the step (b) utilizes an iterative minimization process and further includes iteratively applying the segmented anatomy prior with the CBCT image with a reducing impact factor during successive iterations. In some embodiments, the step (b) further includes iterative alternations between a projection onto convex sets and a total variation minimization.

In some embodiments, the total minimization comprises an adaptive descent projection onto convex sets algorithm. The total minimization can comprise a 11 norm minimization of the gradient image of the CBCT image.

In some embodiments, the first body comprises the thoracic region, and the segmented anatomy prior includes at least one of soft tissue, the lungs and airways, pulmonary details and bone anatomy.

Preferably, the impact factor is geometrically reduced between iterations.

### BRIEF DESCRIPTION OF THE DRAWINGS

Embodiments of the invention will now be described, by way of example only, with reference to the accompanying drawings in which:

FIG. 1 illustrates the anatomy segmentation process to provide a reference image used to adaptively suppress over-smoothing in the TV minimisation process;

FIG. 2 illustrates a flow chart illustrating the algorithm of the preferred embodiment;

FIG. 3 illustrates the selected region f_{SNR} for calculating SNR and CNR;

FIG. 4 illustrates the ground truth image and comparative processing for a digital phantom. The comparative images include FDK, ASD-POCS, PICCS and AACS reconstructed images of the digital phantom. The tumour in each image is highlighted by an arrow and by the sagittal zoom in.

FIG. 5 illustrates the mean absolute differences (MAD) of the reconstructed phantom images, with a lower MAD indicating a more accurate reconstruction of the ground truth.

FIG. 6 illustrates the structural similarity index (SSIM) of the reconstructed phantom images, with a higher SSIM indicating a more accurate reconstruction of the ground truth.

FIG. 7 illustrates the FDK, ASD-POCS, PICCS and AACS reconstructed images of the patient scan. The tumor in each image is highlighted by an arrow. (C/W=0.015/0.03 mm⁻¹);

FIG. 8 illustrates a graph of the SNR values of the patient images.

FIG. 9 illustrates a graph of the CNR values of the tumor and the bony anatomy in the patient images.

FIG. 10 shows an illustrative graph of the total computation time of each CS based reconstruction for phantom data.

FIG. 11 shows an illustrative graph of the total computation time of each CS based reconstruction for patient data. The total computation time was calculated as the sum of the time spent on the SART and TV gradient calculations, as well as the anatomy segmentation operation in the case of AACS. The number of iterations required for each reconstruction is also shown.

FIG. 12 illustrates a pseudo code listing of the AACS algorithm.

### DETAILED DESCRIPTION

The preferred embodiment provides for the use of a 4D CBCT anatomy segmentation prior that can considerably improve 4D CBCT image reconstruction. The preferred embodiment provides a novel CS based thoracic 4D CBCT image reconstruction algorithm that improves on the blurry anatomy and low computational efficiency of conventional TV minimization methods. The preferred embodiment, referred as the anatomical-adaptive compressed sensing (AACS) algorithm, is based on the ASD-POCS framework, but with a novel anatomical-adaptive TV minimization component that utilizes a thoracic 4D CBCT anatomy segmentation method. The theory, implementation and performance evaluation of AACS is described hereinafter. The AACS is demonstrated with the reconstructions of a digital phantom and a patient scan, and compared qualitatively as well as quantitatively to FDK, ASD-POCS, and PICCS. Finally, the limitations and potential future developments of AACS are discussed.

The Feldkamp-Davis-Kress (FDK) reconstruction algorithm currently used for thoracic four-dimensional cone-beam computed tomography (4D CBCT) reconstruction suffers from noise and streaking artefacts due to projection under-sampling. Although compressed sensing (CS) based algorithms can significantly reduce noise and streaking in images reconstructed from under-sampled datasets via total-variation (TV) minimization, they are prone to over-smoothing anatomical details and are computationally inefficient. To overcome these disadvantages, the preferred embodiment provides a new CS based algorithm which exploits the general anatomical knowledge of the thoracic region to preserve anatomical details. The proposed algorithm, referred as the anatomical-adaptive compressed sensing (AACS) algorithm, utilizes the adaptive-steepest-descent projection-onto-convex-sets (ASD-POCS) optimization framework, but incorporates an additional anatomy segmentation step in every iteration. The anatomy segmentation is used as a prior to adaptively suppress TV minimization at anatomical structures of interest and thus avoid over-smoothing. AACS. The results are validated using a digital phantom and a real patient scan, and compared to FDK, ASD-POCS, and the prior image constrained compressed sensing (PICCS) algorithm. For the phantom case, the AACS reconstruction was quantitatively shown to be the most accurate as indicated by the mean absolute difference and the structural similarity index between the reconstructed image and the ground truth. For the patient case, AACS not only resulted in the highest signal-to-noise ratio (i.e. the lowest level of noise and streaking), but also the highest contrast-to-noise ratios for the tumor and the bony anatomy (i.e. the best visibility of anatomical details). Overall, AACS was much less prone to over-smoothing anatomical details compared to ASD-POCS, and did not suffer from residual noise/streaking and motion blur migrated from the prior image like PICCS. AACS was also found to be more computationally efficient than both ASD-POCS and PICCS, with a reduction in computation time of over 50% compared to ASD-POCS. The significant improvement in image quality and computational efficiency makes AACS promising for future clinical use.

Methods: The Theory of AACS

In the following sections, the iterative framework, the novel anatomical-adaptive TV minimization component, and the anatomy segmentation method in AACS are discussed. Finally, the implementation of AACS is summarized.

The Iterative Framework

The AACS algorithm utilizes the ASD-POCS iterative framework, which is a constrained optimization method for solving a solution image with minimized TV. Denoting the image as \vec{f}, the measured projection data as \tilde{p} and the forward projection operator as R, the ASD-POCS framework is defined as:

$${{f}^{->}}_{ASD - POCS} = {argmin}_{{f}^{->}} TV ( {f}^{->} ) , s . t . ‖ R {f}^{->} - \tilde{p} ‖ \leq ε , {f}^{->} \geq 0. \qquad ( 1 )$$

where ε is the maximum discrepancy between the projection data and the forward projections of the solution image, and TV is defined as:

$$TV ( {f}^{->} ) = {\sum }_{r . s . t} \vert ∇ {f}_{r . s . t} ‖ , \qquad ( 2 )$$

where

$$‖ ∇ {f}_{r . s . t} \vert = \sqrt{{( {f}_{r . s . t} - {f}_{r - 1. s . t} )}^{2} + {( {f}_{r . s . t} - {f}_{r . s - 1. t} )}^{2} + {( {f}_{r . s . t} - {f}_{r . s . t - 1} )}^{2}} . \qquad ( 3 )$$

and where r, s, t are the three dimensional spatial indices.

The implementation of ASD-POCS consists of iterative alternations between two major components —the POCS component and the TV minimization component.

The POCS component enforces the positivity constraint \vec{f}≧0 and the data fidelity constraint ∥\vec{f}−\tilde{p}∥<ε in every iteration, and is realized by applying either an algebraic reconstruction technique (ART) or a simultaneous algebraic reconstruction technique (SART) step. Following every POCS step, TV is minimized by a few iterations of gradient steepest-descent (GSD) steps. The TV GSD step is approximated by that derived in the literature (Niu and Zhu, 2012).

$${[ - {∇}_{{f}^{->}} TV ( {f}^{->} ) ]}_{r . s . t} = \frac{{f}_{r - 1. s . t} + {f}_{r . s - 1. t} + {f}_{r . s . t - 1} - 3 {f}_{r . s . t}}{\sqrt{δ + {( {f}_{r . s . t} - {f}_{r - 1. s . t} )}^{2} + {( {f}_{r . s . t} - {f}_{r . s - 1. t} )}^{2} + {( {f}_{r . s . t} - {f}_{r . s . t - 1} )}^{2}}} + \frac{{f}_{r - 1. s . t} - {f}_{r . s . t}}{\sqrt{\begin{matrix}δ + {( {f}_{r - 1. s . t} - {f}_{r . s . t} )}^{2} + \\ {( {f}_{r + 1. s . t} - {f}_{r + 1. s - 1. t} )}^{2} + {( {f}_{r + 1. s . t} - {f}_{r + 1. s . t - 1} )}^{2}\end{matrix}}} + \frac{{f}_{r . s + 1. t} - {f}_{r . s . t}}{\sqrt{\begin{matrix}δ + {( {f}_{r . s - 1. t} - {f}_{r - 1. s + 1. t} )}^{2} + \\ {( {f}_{r . s - 1. t} - {f}_{r . s . t} )}^{2} + {( {f}_{r . s - 1. t} - {f}_{r . s + 1. t - 1} )}^{2}\end{matrix}}} + \frac{{f}_{r . s . t - 1} - {f}_{r . s . t}}{\sqrt{\begin{matrix}δ + {( {f}_{r . s . t + 1} - {f}_{r - 1. s . t + 1} )}^{2} + \\ {( {f}_{r . s . t + 1} - {f}_{r . s - 1. t - 1} )}^{2} + {( {f}_{r . s . t + 1} - {f}_{r . s . t} )}^{2}\end{matrix}}} . \qquad ( 4 )$$

where δ is a small positive number to avoid singularities in the calculation, and can be set to the machine epsilon (≈2×10⁻¹⁶). At the end of each iteration, the POCS step size and the TV minimization step size are adaptively reduced to achieve balance between the two components. Detailed descriptions of the step size reduction schemes can be found in Sidky and Pan (2008).

Anatomical-Adaptive TV (AATV) Minimization:

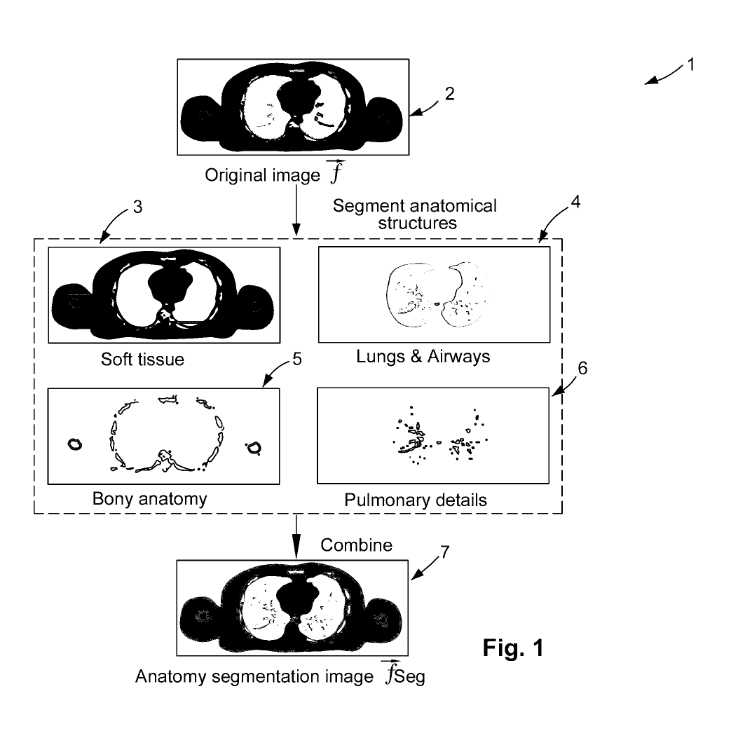

Conventional TV is essentially the sum of pixel intensity variations (cf. equation (2)) regardless of whether the intensity variation of a pixel is attributed to noise/streaking or the presence of anatomical structures. In other words, the TV minimization component in ASD-POCS cannot distinguish between noise/streaking and anatomical structures, and thus often causes loss of image details due to over-smoothing. Such loss of image details can be spared by exploiting a “modified TV” that minimizes the contribution of intensity variations from anatomical structures. In AACS, this modified TV term is referred to as the anatomical-adaptive TV (AATV), and is defined as:

$$AATV ( {f}^{->} ) = {\sum }_{r . s . t} ( ‖ ∇ {f}_{r . s . t} \vert - λ \vert ∇ {f}_{Seg . r . s . t} ‖ ) . 0 \leq λ \leq 1 , \qquad ( 5 )$$

where the subscript “Seg” refers to the anatomy segmentation image, \vec{f}_{Seg}, and where λ is an impact factor described below. The anatomy segmentation image, as illustrated in FIG. 1, is a “simplified sketch” of the updated solution image \vec{f} in each iteration, with only the “major anatomical structures” (i.e. soft tissue, lungs/airways, bony anatomy, and pulmonary details) segmented from \vec{f} and represented by their likely attenuation coefficients. The acquisition of the anatomy segmentation image \vec{f}_{Seg}, from \vec{f} is the key to the AACS algorithm, and is discussed in detail below. Since an ideal anatomy segmentation image contains only the major anatomical structures, the ∥\vec{∇f}_{Seg,r,s,t}∥, term can be viewed as an anatomy segmentation prior, subtraction of which should in principle remove the contribution of anatomical related intensity variations to AATV(\vec{f}). In other words, AATV minimization is expected to adaptively suppress image smoothing at anatomical structures of interest compared to conventional TV minimization. However, due to the inferior image quality of 4D CBCT, the anatomy segmentation image is often only a reasonable estimate instead of a highly accurate representation of the anatomy. Therefore, an “AATV impact factor” λ, is introduced to weight the impact level of the anatomy segmentation prior (a higher λ indicates a higher impact of the anatomy segmentation prior). In practice, λ is gradually reduced from unity as the algorithm iterates, so that the impact of the anatomy segmentation prior is greater in early iterations and lower when close to convergence. This λ reduction scheme allows the anatomy segmentation prior to render considerable improvement in the reconstruction performance while not biasing the solution towards inaccuracies in the segmentation image. The reduction scheme for λ is discussed below.

In a similar way to the TV minimization in ASD-POCS, AATV can be minimized by applying a few iterations of GSD steps. The AATV GSD step can be derived from taking the negative gradient of equation (5) with respect to \vec{f}

∇_{\vec{f}}AATV(\vec{f})=−∇TV(\vec{f})+λ∇_{\vec{f}}TV(\vec{f}_{Seg}) (6)

It can be seen from equation (6) that the AATV GSD step is the combination of the TV GSD step, −∇_{\vec{f}}TV(\vec{f}), and an anatomy segmentation prior term, λ−∇_{\vec{f}}TV(\vec{f}_{Seg}), which suppresses image smoothing at anatomical structures of interest.

In practice it is difficult to compute the AATV GSD step via equation (6), because ∇_{\vec{f}}TV(\vec{f}_{Seg}) cannot be directly calculated as there is no explicit expression of \vec{f}_{Seg} in terms of \vec{f}. However, by assuming that \vec{f}_{Seg} is in general not sensitive to small intensity variations in \vec{f}, i.e. assuming that the anatomy segmentation method is reasonably robust to noise, equation (6) can be approximated by:

−∇_{\vec{f}}AATV(\vec{f})≈−∇_{\vec{f}}TV(\vec{f})+λ∇_{\vec{f}}_{_{SegTV(}}\vec{f}_{Seg}). (7)

where the ∇_{\vec{f}}TV (\vec{f}_{Seg}) term has been replaced by

$$∇ {\to }_{{f}_{Seg}} TV ( {\vec{f}}_{Seg} ) ,$$

so that both terms in equation (7) can be explicitly calculated using equation (4).

Anatomy Segmentation

As shown in FIG. 1, the anatomy segmentation image 7 is obtained by segmenting the four major anatomical structures—soft tissue 3, lungs/airways 4, bony anatomy 5, and pulmonary details 6—from the updated solution \vec{f} in every iteration. For the purpose of AACS reconstruction, a reasonable anatomy estimation is sufficient to considerably improve the reconstruction performance. Thus, the anatomy segmentation method utilized in AACS is mainly based on simple intensity thresholding and pixel connectivity strategies, and does not aim for a perfectly accurate segmentation. The step-by-step segmentation details are given below.

1. Soft tissue: The soft tissue 3 is segmented by pixels with attenuation coefficients higher than the soft tissue attenuation threshold I_{soft}. Then, only the largest connected area in the thresholded mask is labeled as soft tissue, so that noise/streaking exterior to the patient and fine details inside the lungs are excluded. A value used for I_{soft}≈0.009 mm^{-l}.

2. Lungs/airways: Once the soft tissue has been segmented, the rest of the low attenuation regions belong to either the background or the lungs/airways 4. To eliminate the background, for every axial slice, a background removal operator starts multiple searches from the pixels on the four boundaries, each search moving towards the center along the anterior-posterior (AP) or left-right (LR) direction. Once a search encounters the soft tissue region, pixels preceding the first soft tissue pixel are identified as background and eliminated from the image. Having removed the background, the rest of the low attenuation regions are attributed to the lungs/airways and possibly some noise/streaking outside the lungs. Since the lungs and airways are in general much larger in volume than noise/streaking, excluding regions with connected volume less than the lung/airway volume threshold V_{Lung} renders a noise- and streak-reduced lung/airway segmentation. A value of V_{Lung}≈10 mm³ is suitable.

3. Pulmonary details: Pulmonary details 6 refer to any contrast objects inside the lung, e.g. tumors, vessels, bronchus walls. In general, pulmonary details are similar in attenuation coefficients to soft tissue. However, pulmonary details often suffer from loss of contrast either due to their small sizes or motion artifacts. Thus, compared to the soft tissue attenuation threshold, a slightly lower threshold value I_{Pulmonary} ≈0.008 mm⁻¹ was used to segment pulmonary details inside the lungs.

Bony Anatomy:

The bony anatomy 5 can be roughly segmented by pixels with attenuation coefficients higher than the bone attenuation threshold Iₒₙₑ. A suitable value for I_{Bone}≈0.016 mm⁻¹. However, the thresholded image often retains streaking artifacts due to the inferior image quality of the 4D images. To exclude the majority of the streaking artifacts, a reference segmentation of the bony anatomy was first acquired by segmenting the 3D FDK image. Since the 3D FDK image is relatively streak-free and does not suffer from significant motion artifacts in the bony anatomy, the attenuation thresholded result alone is sufficient to render an accurate reference segmentation. A “search region” was then constructed to account for respiratory motion by extending the reference segmentation in the coronal, sagittal, and axial directions by approximately 2 mm, 2 mm, and 5 mm, respectively. Finally, the attenuation thresholded segmentation of the 4D image was masked with the search region to give a more streak-free segmentation of the bony anatomy.

Combine Segmentations:

Each of the anatomical structures 3-6 is assigned a single representative attenuation coefficient, and combined to give the anatomy segmentation image 7, as illustrated in FIG. 1. The 3D FDK image offers a reliable estimate of the representative attenuation coefficients since it has much better image quality than the 4D images, leaving aside motion artifacts. Thus, the representative attenuation coefficients of the soft tissue, lungs/airways, and bony anatomy are estimated by the mean attenuation coefficients of their segmentations in the 3D FDK image. The pulmonary details are represented by the soft tissue attenuation coefficient.

Implementation of AACS:

AACS can be implemented by a constrained optimization algorithm solving for a solution with minimized AATV.

$${{f}^{->}}_{AACS} = {argmin}_{{f}^{->}} AATV ( {f}^{->} ) . s . t . ‖ R {f}^{->} - \tilde{p} ‖ \leq ε . {f}^{->} \geq 0. \qquad ( 8 )$$

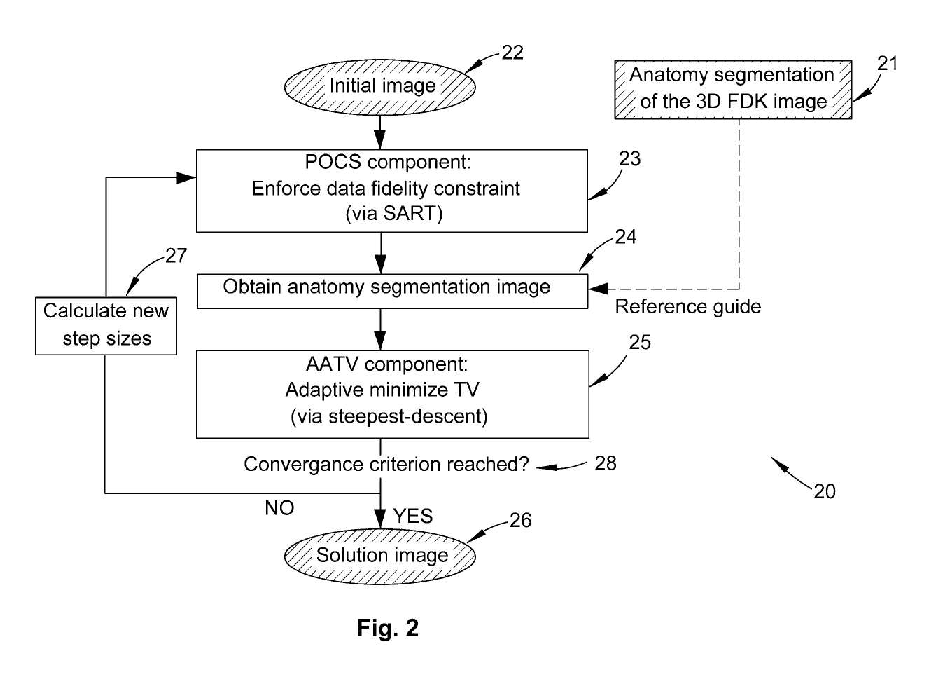

with AATV defined in equation (5). The implementation of AACS is summarized 20 in FIG. 2. Prior to the iterative process, the 3D FDK image is reconstructed, and its anatomy segmentation image is acquired 21 as a reference guide to the anatomy segmentation of the 4D images. Then, the iterative process is usually initialized from either a zero image or a FDK image 22. Each iteration starts with a POCS component (realized as SART) to enforce the data fidelity constraint 23. Then, the anatomy segmentation image of the POCS updated image is acquired 24, with which AATV of the POCS updated image is minimized 25 via applying a few consecutive steps (typically ≈20) of equation (7). At the end of each iteration, depending on whether the convergence criterion is reached or not, the iterative process either converges and returns the POCS updated image 26, or calculates new POCS/AATV step sizes 27 and the AATV impact factor λ before continuing to the next iteration. The convergence criterion of AACS is when the norm of the change of image in one iteration is smaller than a specified magnitude. A pseudo code implementation is shown in FIG. 9 and discussed further below.

The selection schemes for the POCS and AATV step sizes are described in detail in Sidky and Pan (2008). The AATV impact factor λ was initialized to be unity, and was gradually reduced with the AATV step size a, using a heuristic update scheme

$${λ}^{( k )} = {[ \frac{{α}^{( k )}}{{α}^{( 1 )}} ]}^{1 / γ} . \qquad ( 9 )$$

where k is the current iteration number, and γ>0 is a predefined parameter determining how rapidly λ is reduced. A higher γ slows the reduction of λ, resulting in an overall greater impact of the anatomy segmentation prior. For example, γ=4 was used throughout.

Performance Assessment

AACS was applied to both a digital phantom dataset and a clinical patient dataset for performance evaluation. For comparison, both datasets were also reconstructed with FDK, ASD-POCS and PICCS.

Phantom Data

A realistic ten-phase 4D thoracic phantom was simulated using the XCAT digital phantom (Segars et al, 2010). Ten ground truth images were generated with 512² voxels ((0.88 mm)² voxel size) in 128 axial slices (2 mm slice thickness). A spherical tumor with a diameter of 12 mm was placed in the lower lobe of the right lung near the mediastinum. The scan geometry was chosen according to the Varian On-Board Imager (Varian Medical Systems, Palo Alto, Calif.) half-fan acquisition mode Lu et al (2007). In order to exclude any respiratory binning related motion artifacts and only focus on the effect of the reconstruction algorithms, the projections were generated from forward projecting the ten discrete ground truth images instead of a continuously breathing phantom, and each respiratory phase was later reconstructed with the projections that were forward projected from the corresponding ground truth image. The scan duration was 250 s, in which 50 respiratory cycles of 5 s were included. A total of 1200 half-fan projection images were generated, covering an angular range of 360° and each with a dimension of 256×128 and pixel size of 1.552×3.104 mm². Similar to that adopted by Bergner et al (2010), Poisson noise modeling 30000 photons per ray was added to the projection images. The reconstruction resolution was the same as that of the ground truth images.

Patient Data

AACS was also applied to a clinical scan from a stereotactic body radiation therapy patient. The scan was acquired with the Elekta Synergy (Elekta Oncology Systems Ltd, Crawley, UK) full-fan acquisition mode. Due to the limited field of-view (FOV) of full-fan acquisition, the left lung was truncated in the reconstructed image. The scan duration was approximately 4 minutes, in which 93 respiratory cycles (free breathing) were included. The scan contains a total of 1340 projection images, covering an angular range of 200°. The projection images were sorted into ten phase bins using a projection intensity based sorting method (Kavanagh et al, 2009). The reconstructed image contained 128 axial slices with a slice spacing of 2 mm, each slice containing 5122 voxels with a voxel size of (0.5 mm)2. The original projection image dimension was 5122 with a pixel size of (0.8 mm)2, and was down-sampled in the longitudinal direction to 512×128 with a pixel size of 0.8×3 2 mm2 in order to match the reconstruction resolution near isocenter ((0.8, 3.2) mm×SAD/SID≈(0.5, 2) mm). Projection down-sampling saves unnecessary computational time and reduces Poisson noise without causing observable loss in spatial resolution (Cropp, 2011).

Reconstruction Parameters

The FDK algorithm is a simple filtered backprojection method and requires no input parameter. The reconstruction filter was the standard RamLak kernel.

For the ASD-POCS reconstruction, the initial TV minimization step size, α, was set to 0.05 (phantom case) and 0.1 (patient case). The TV reduction factor, α_{red}, was set to 0.8 for both the phantom and patient cases. The threshold for TV reduction, rₘₐₓ, was set to 0.9 (phantom case) and 0.8 (patient case). The residual error tolerance for TV reduction, tol, was set to 0.11 (phantom case) and 1.25 (patient case). The POCS reduction factor, β_{red}, was set to 0.99 for both the phantom and patient case.

For the PICCS reconstruction, the 3D motion blurred FDK image was used as the prior image with a prior weighting factor λ_{PICCS} of 0.5 as adopted by Bergner et al (2010). There is no step size reduction scheme for PICCS, and therefore α was set to be 0.025 (phantom case) and 0.004 (patient case), which is relatively small compared to that of ASD-POCS.

For the AACS reconstruction, the anatomy segmentation prior allows the use of a larger initial TV minimization step size without over-smoothing anatomical details. Thus, a was set to 0.2 (phantom case) and 0.4 (patient case). Since this renders much more rapid removal of noise and streaking artifacts than ASD-POCS, a smaller TV reduction factor of 0.4 was used to accelerate convergence without sacrificing image quality. The other parameters were set to be the same as that of ASD-POCS.

For all three iterative algorithms, the 4D FDK image of the corresponding phase was used as the initial image. The convergence criterion was the norm of the image change in one iteration dropping below 2×10⁻⁴ mm⁻¹ for the phantom case and 2.5×10⁻⁴ mm⁻¹ for the patient case. Twenty steps were used for the GSD minimization component, applying which ASD-POCS minimizes conventional TV (cf. equation (1)), AACS minimizes AATV (cf. equation (8)), and PICCS minimizes an objective function combining the conventional TV and the similarity between \vec{f} and the prior image \vec{f}_{Prior}, viz.

$${{f}^{->}}_{PICCS} = {argmin}_{{f}^{->}} [ ( 1 - {λ}_{PICCS} ) TV ( {f}^{->} ) + {λ}_{PICCS} TV ( {f}^{->} - {{f}^{->}}_{Prior} ) ] . \\ s . t . ‖ R {f}^{->} - \tilde{p} ‖ \leq ε . {f}^{->} \geq 0. \qquad ( 10 )$$

The POCS step was realized as SART for the phantom case. For the patient case, SART was susceptible to truncation artifacts due to the limited FOV, and therefore the POCS step was realized as FDK backprojection of the difference projection instead (Zeng and Gullberg, 2000).

The reconstructions were computed on a dual Intel Xeon E5-2687W CPU with a clock speed of 3.1 GHz each. All the reconstructions were performed using in-house MATLAB codes, with the FDK backprojection, forward projection, and SART modules from the Reconstruction Toolkit developed by Rit et al (2014).

Image Quality Metrics

The reconstruction accuracy of the digital phantom was assessed by the similarity between the reconstructed image, \vec{f} and the ground truth (GT) image, \vec{f}_{GT}, using two metrics. The first metric, the mean absolute difference (MAD), measures similarity relative to ground truth on a pixel-by-pixel basis, and is mathematically defined by

$$MAD = \frac{1}{N} {\sum }_{j = 1}^{N} \vert {f}_{j} - {f}_{GT , j} \vert , \qquad ( 11 )$$

where N is the number of image pixels. A lower MAD indicates higher similarity with the ground truth image, hence better image quality. The second metric, the structural similarity (SSIM) index, measures human visual perception to degradation of structural information, and is more clinically relevant than MAD. SSIM ranges from 0 to 1, with a higher value indicating higher similarity with the ground truth image. A detailed definition of SSIM can be found in (Wang et al, 2004). In this embodiment the mean SSIM value over all axial slices was used.

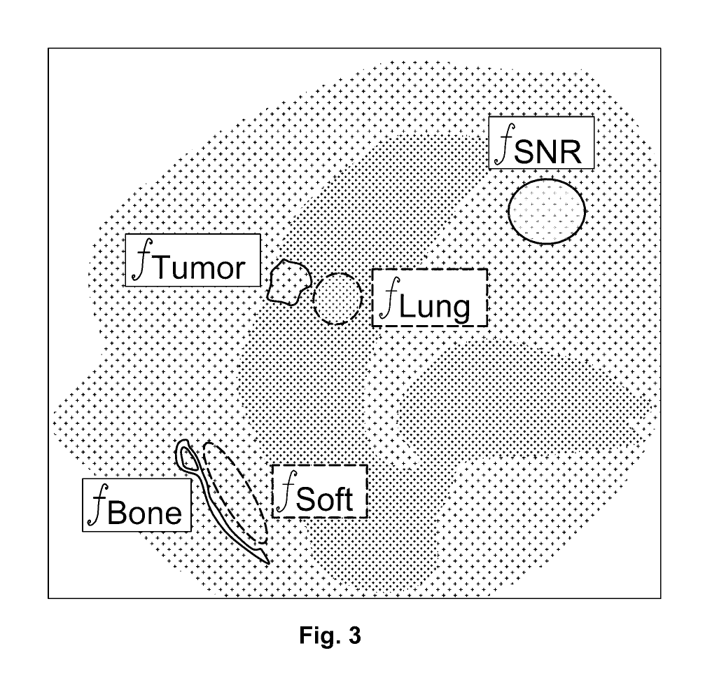

The image quality of the reconstructed patient image was assessed by the level of noise and streaking and the visibility of anatomical details. The level of noise and streaking was quantified by the signal-to-noise ratio (SNR). SNR was calculated over a selected uniform region, the set of pixel values belonging to which is denoted by f_{SNR} (cf. FIG. 3), using the following formula

$$SNR = \frac{Mean ( {f}_{SNR} )}{SD ( {f}_{SNR} )} . \qquad ( 12 )$$

where SD denotes standard deviation. The visibility of anatomical details was quantified by the contrast-to-noise ratio (CNR) of the tumor and the bony anatomy. To calculate CNR, the tumor and the part of the scapula in the axial slices where the tumor was visible were first manually delineated from the reconstructed image. The scapula was chosen for bone CNR calculation because it can be clearly delineated in all reconstructed images. A lung region near the tumor and a soft tissue region near the scapula were selected as the “background”. Denoting the sets of pixel values of the tumor, scapula, lung, and soft tissue as f_{Tumor}, f_{Bone}, f_{Lung}, and f_{soft} (cf. FIG. 3), the tumor and bone CNRs can be calculated by

$$\begin{matrix}{CNR}_{Tumor} = \frac{Mean ( {f}_{Tumor} ) - Mean ( {f}_{Lung} )}{\sqrt{Variance ( {f}_{Tumor} ) + Variance ( {f}_{Lung} )}} . & ( 13 ) \\ {CNR}_{Bone} = \frac{Mean ( {f}_{Bone} ) - Mean ( {f}_{Soft} )}{\sqrt{Variance ( {f}_{Bone} ) + Variance ( {f}_{Soft} )}} . & ( 14 )\end{matrix}$$

Results

Image Quality Phantom Data:

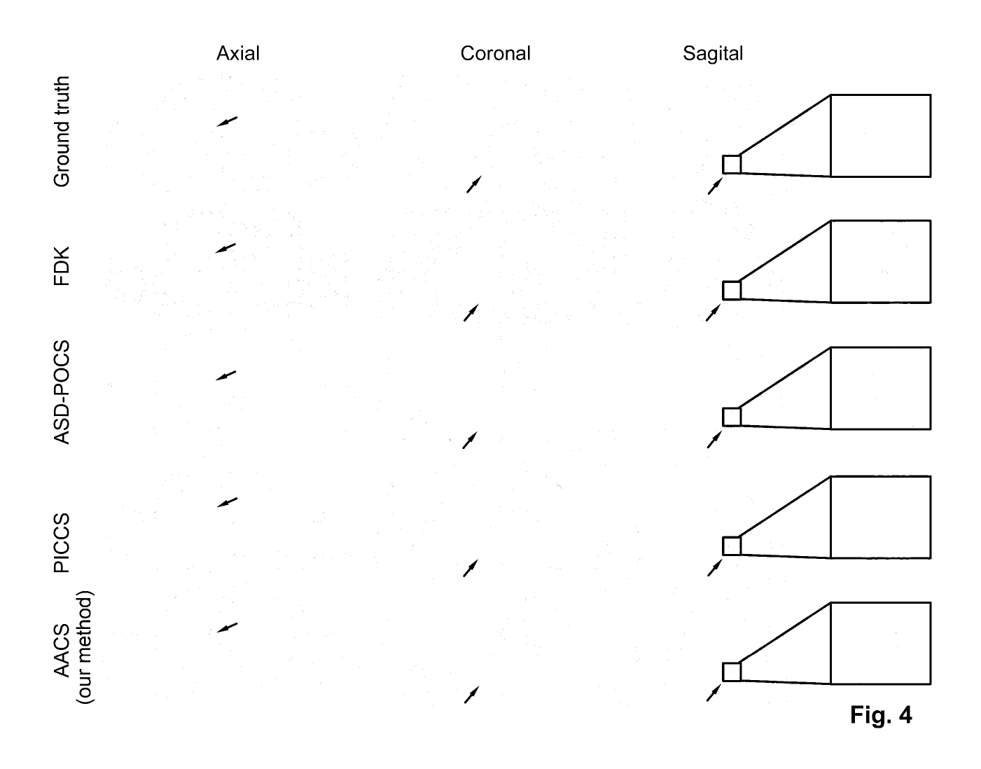

The 20% phase (mid-exhale) of the digital phantom, reconstructed with 50 half-fan projection images, was chosen for comparing reconstruction algorithms. The FDK, ASD-POCS, PICCS, and AACS reconstructed images and the ground truth are displayed in FIG. 4.

In terms of noise and streaking, all three CS based algorithms (ASD-POCS, PICCS, and AACS) performed significantly better than FDK. The ASD-POCS image has the lowest level of noise and streaking, closely followed by the AACS image, in which minor streaking artifacts can be observed but barely influence the visibility of any details. Among the three CS based algorithms, the PICCS image contains the most noise and streaking artifacts inherited from the prior image.

In terms of blurring, the ASD-POCS image shows the worst contrast and sharpness of the bony anatomy and pulmonary details due to over-smoothing, which is expected as conventional TV minimization smooths all intensity variations indistinguishably. The PICCS image has a much improved overall contrast and sharpness compared to the ASD-POCS image. In particular, the bony anatomy in the PICCS image is the clearest among all four reconstructed images. Nevertheless, the contrast of the pulmonary details is slightly worse than that of FDK, which is likely due to motion blur inherited from the prior image. AACS is much less prone to over-smoothing compared to ASD-POCS, and does not suffer from motion blur inherited from the prior image like PICCS. AACS thus rendered the best contrast of pulmonary details. The bony anatomy appears to be slightly blurrier in the AACS image compared to the PICCS image, but is considerably clearer than that in the FDK and ASD-POCS images.

The sagittal zoom in shows that AACS rendered the most accurate and distinct reconstruction of the tumor shape. The tumor contour in the FDK image is corrupted by noise and streaking artifacts. ASD-POCS “over-polished” the edges, resulting in a reasonably defined but blunt contour. PICCS was unable to restore a distinct tumor contour due to motion blur.

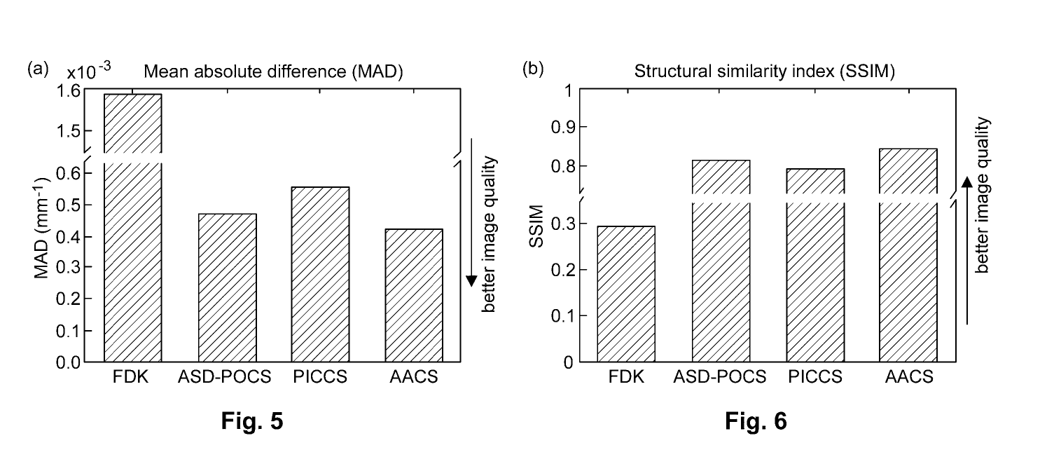

In summary of the qualitative assessment, the AACS image exhibits the best overall image quality, as its low noise/streaking level and high contrast/sharpness gave the best visibility of most details. This conclusion was quantitatively verified using MAD and SSIM. FIG. 5 shows that the AACS image gave the lowest MAD (4.3×10⁻⁴ mm⁻¹), followed by ASD-POCS (4.8×10⁻⁴ mm⁻¹), PICCS (5.5×10⁻⁴ mm⁻¹), then FDK (15.9×10⁻⁴ mm⁻¹). FIG. 6 shows that the AACS image gave the highest SSIM (0.85), followed by ASD-POCS (0.81), PICCS (0.80), then FDK (0.30). These results demonstrate that AACS rendered the most accurate reconstruction of the ground truth from both a pixel-by-pixel aspect, i.e. the lowest MAD, and a visual perception aspect, i.e. the highest SSIM. It should be noted that since both MAD and SSIM are global image quality metrics, they are in general not predominated by the visibility of fi image details, which accounts for the main qualitative differences between the ASD-POCS, PICCS, and AACS images. In other words, despite the small differences in MAD and SSIM between the three CS based reconstructions, these small quantitative diff are in fact qualitatively significant as inferred from visual inspection of FIG. 4.

Patient Data

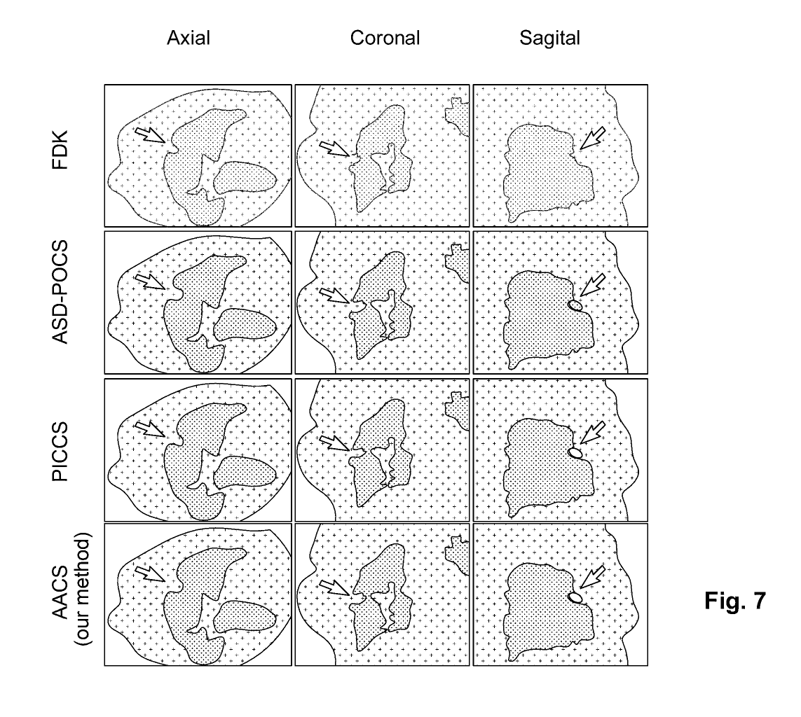

The FDK, ASD-POCS, PICCS, and AACS images of the 20% phase (mid-exhale) of the patient scan, reconstructed with 73 full-fan projection images, are displayed in FIG. 7. All three CS based algorithms rendered significant improvements in image quality compared to FDK, especially in terms of noise and streaking reduction. Among the three CS based reconstructions, the PICCS image exhibits the most noise and streaking artifacts, most likely inherited from the prior image. The ASD-POCS image is smoother than the PICCS image, but suffers from slight blurring of the bony anatomy. The AACS image not only exhibits the least noise and streaking artifacts, but also shows the best contrast and sharpness especially of the bony anatomy. Nevertheless, it is noteworthy that the vertebra appears to be slightly clearer in the PICCS image than in the AACS image. This is expected since the reconstruction of nearly stationary anatomies such as the vertebra is barely affected by motion blur, and thus benefits the most from the use of the 3D motion blurred prior image in PICCS.

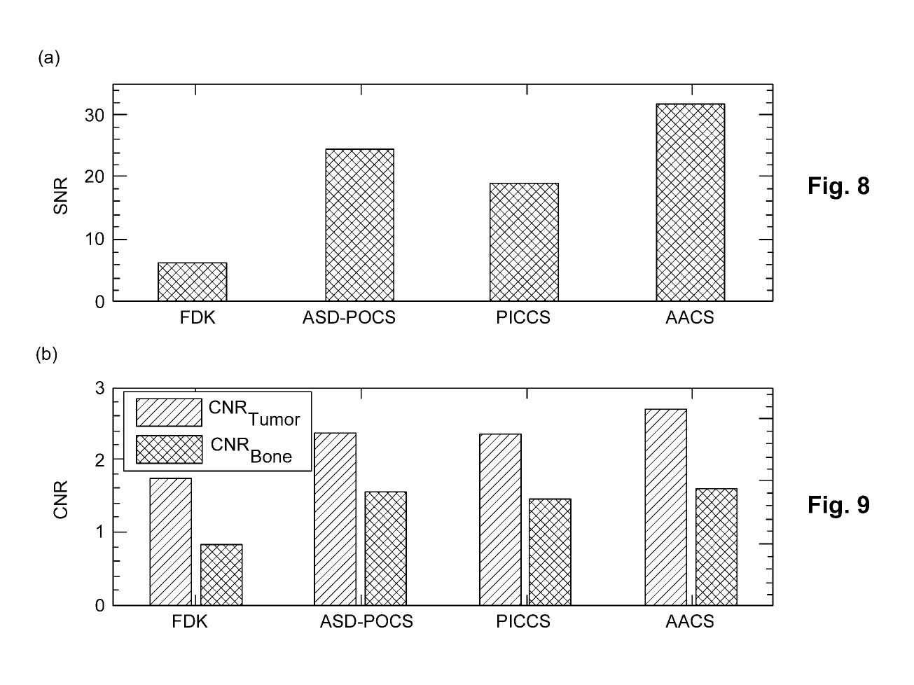

SNR and CNR were used to quantify the level of noise and streaking and the visibility of the anatomical details in the reconstructed image, respectively. FIG. 8 shows that the AACS image has the highest SNR (31.9), followed by ASD-POCS (24.5), PICCS (19.4), then FDK (6.2), corroborating the qualitative analysis inferred from visual inspection of FIG. 6. FIG. 9 shows that AACS also produced the highest CNR values for both the tumor (2.73) and the bony anatomy (1.61), followed by ASD-POCS (2.39, 1.55), PICCS (2.39, 1.47), then FDK (1.74, 0.86). This indicates that anatomical details can be the most clearly identified in the AACS image due to the high contrast and low level of noise and streaking. It is also worth mentioning that although the ASD-POCS image and the PICCS image have very similar CNRs, they show notably different qualitative characteristics such that the former exhibits less noise and streaking artifacts while the latter exhibits slightly better contrast and sharpness of the anatomy.

Computational Efficiency

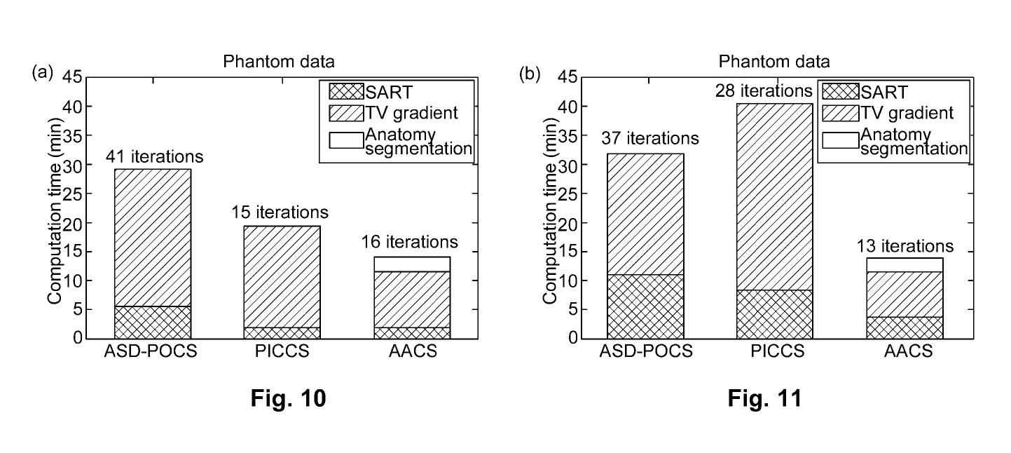

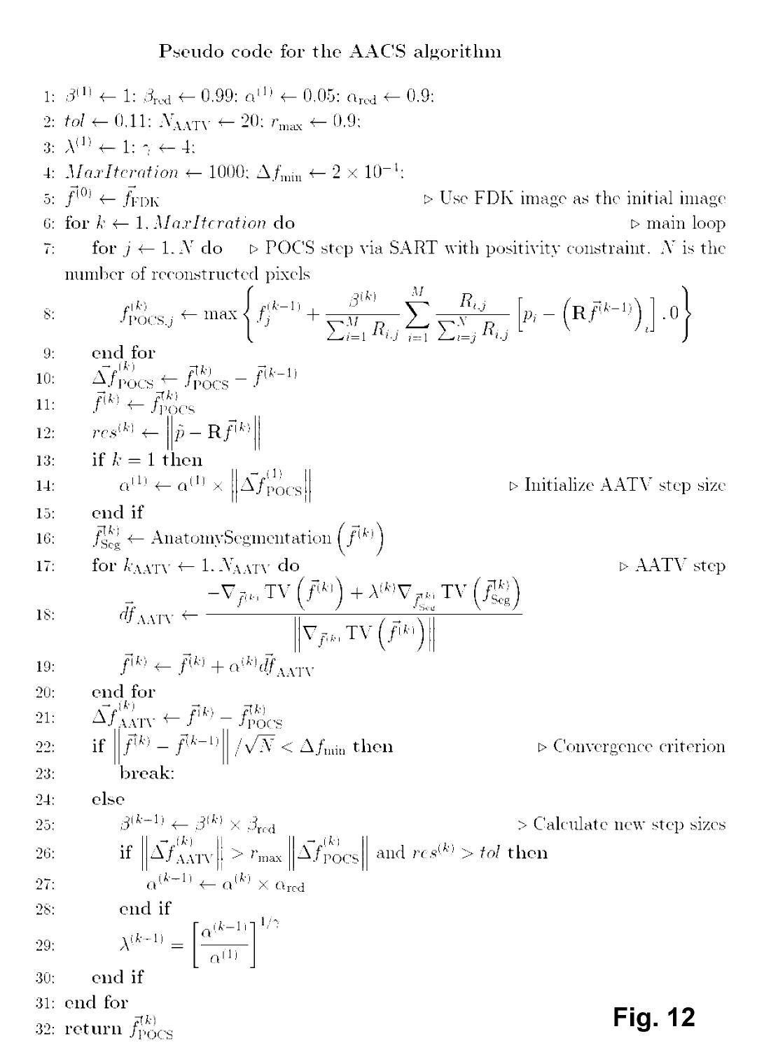

The computational efficiency of AACS was compared to that of the other CS based iterative algorithms, i.e. ASD-POCS and PICCS, by the total computation time required for each reconstruction. The total computation time was recorded as the sum of the time spent on the major operations—SART, TV gradient calculation, and anatomy segmentation (AACS only). FIG. 10 displays the computation time and the number of iterations required for each CS based reconstruction for the phantom case and FIG. 11 shows the efficiency for the patient cases. It should be noted that the computation time occupied by each operation may vary depending on factors such as code optimizations and computer hardware specifications.

ASD-POCS required the most iterations to converge for both the phantom data (41 iterations) and patient data (37 iterations), which is to be expected as it does not exploit any prior knowledge to accelerate convergence. PICCS required only one less iteration to converge compared to AACS for the phantom data (15 vs. 16), but required more than twice as many iterations for the patient data (28 vs. 13). In theory, PICCS is expected to converge faster than AACS since the prior image constraint is stricter than the anatomy segmentation prior. However, in this study PICCS was implemented with a constant GSD step size as adopted in Leng et al (2008b) and Bergner et al (2010), which is most likely responsible for the delayed convergence. Other step size selection schemes and optimization frameworks have been proposed for PICCS (Lauzier et al, 2012), and may improve convergence performance than that found in this study.

In terms of total computation time, AACS is the most efficient among all three CS based algorithms, taking only approximately 15 minutes to converge for both the phantom and patient data. This is over 50% more efficient than ASD-POCS (for both phantom and patient data), and approximately 25% (phantom) and 70% (patient) more efficient than PICCS. For the reconstruction dimension used in this study, which is typical for clinical thoracic CBCT, the TV gradient calculation is relatively computationally expensive compared to SART and anatomy segmentation, and accounts for the majority of the total computation time. For a 20-step GSD minimization component per main iteration, the number of TV gradient calculation operations required for each algorithm is: 20 for ASD-POCS, 21 for AACS (one additional calculation for the anatomy segmentation prior, cf. equation (7)), and 40 for PICCS (2×20 since there are two TV terms in the objective function, cf. equation (11)). Consequently, the tradeoff for faster convergence in PICCS is the requirement of considerably more TV gradient calculation operations per iteration than ASD-POCS and AACS, making PICCS more time consuming than that suggested by the number of iterations. In contrast, AACS is much more computationally economical as it achieves faster convergence for the relatively low cost of only one additional TV gradient calculation and one computationally cost-effective anatomy segmentation step per iteration.

Discussion

The preferred embodiment provides a novel CS based thoracic 4D CBCT image reconstruction algorithm, i.e. the AACS algorithm, which overcomes some limitations of conventional CS based algorithms by exploiting the general anatomical knowledge of the thoracic region in the form of an anatomy segmentation prior. As demonstrated by both the phantom and patient cases, the incorporation of the anatomy segmentation prior renders significant improvements in image quality and computational efficiency compared to both ASD-POCS and PICCS. The improved reconstruction performance is attributed to two main advantages of the use of the anatomy segmentation prior. Firstly, the anatomy segmentation prior helps the reconstruction algorithm identify and adaptively preserve anatomical structures of interest in the image smoothing process. Consequently, AACS is able to achieve significant reduction in noise and streaking comparable to that achieved by ASD-POCS, but without apparent loss of contrast and sharpness. Furthermore, the anatomy segmentation prior allows a larger AATV minimization step size to be used for more rapid noise and streaking removal without over-smoothing the anatomical structures, thereby reducing the number of iterations required for a clean reconstruction. Further, since the anatomy segmentation prior is acquired based on “general” anatomical knowledge of the thoracic region, which is applicable to every thoracic 4D CBCT scan, it is much less strict than the prior image constraint employed in PICCS. In other words, AACS is less prone to biasing the solution than PICCS. Consequently, AACS results in a lower level of noise and streaking as well as better contrast than PICCS, as the latter suffers from noise, streaking artifacts, and motion blur inherited from the 3D motion blurred prior image.

In this work, a simple anatomy segmentation method was used for AACS, and was demonstrated to be sufficient to render significant improvements in the reconstruction performance despite the low accuracy of anatomy segmentation of 4D CBCT images. This is firstly because AACS does not strictly enforce similarity between the solution and the anatomy segmentation image, and is therefore reasonably robust to small inaccuracies in the segmentation. Secondly, since the anatomy segmentation image is re-computed in every iteration, the accuracy of the segmentation improves with the quality of the solution image as the algorithm iterates. Nevertheless, a major limitation of this simple anatomy segmentation method is that the segmentation outcome can be sensitive to the selection of segmentation parameters, e.g. intensity and connectivity thresholds. In this work, the segmentation parameters were manually predetermined, which is undesirable in practice as the optimal parameters will in general vary from scan to scan depending on factors such as beam settings and patient sizes. Fortunately, automatic selection of thresholding parameters has been demonstrated for anatomy segmentation of CT images (van Rikxoort et al, 2009; Volpi et al, 2009), and may be incorporated into AACS to facilitate more reliable segmentation outcomes.

We have proposed AACS as a constrained optimization algorithm based on the ASD-POCS framework utilizing AATV minimization in replacement of conventional TV minimization. Nevertheless, the AATV minimization component, which is the key innovation of AACS, can also be easily combined with other optimization strategies to achieve further improvements. For example, the convergence of AACS may be further accelerated by utilizing the accelerated barrier unconstrained optimization framework proposed by Niu and Zhu (2012). The use of the 3D motion blurred prior image in PICCS may also be incorporated into AACS to improve the reconstruction of nearly stationary anatomies such the vertebra.

The use of the anatomy segmentation prior not only results in better image quality, but also reduces the computation time of CS based reconstructions by over 50%. It was found in this study that AACS was able to reconstruct a high resolution and high quality 4D CBCT image in approximately 15 minutes, which is a significant advancement towards the clinical feasibility of iterative reconstructions compared to >30 minutes computation time of conventional CS based reconstructions. Further efficiency gains may be realized through GPU implementation and advanced optimizations of AACS, which may speed up the reconstruction process by a factor of 20 or more (Jia et al, 2010; Tian et al, 2011). This can potentially reduce the computation time even further, thereby facilitating AACS for clinical use.

It will be apparent that, although AACS is presented as an algorithm for thoracic 4D CBCT reconstruction, the core concept of AACS, i.e. exploiting general anatomical knowledge in the form of anatomy segmentation, is not limited to thoracic CBCT scans, and may also be applied to other anatomical regions and imaging modalities.

### CONCLUSION

An effective Anatomical-Adaptive Compressed Sensing (AACS) has been proposed, a CS based thoracic 4D CBCT reconstruction algorithm which exploits the general anatomical knowledge of the thoracic region in the form of an anatomy segmentation prior. Using a phantom and a patient study, we have shown that compared to other CS based algorithms, AACS not only significantly improves image quality in terms of reconstruction accuracy, signal-to-noise ratio, and contrast-to-noise ratio, but also shortens the computation time by over 50%. Further developments can potentially facilitate clinical use of AACS, enabling high quality thoracic 4D CBCT reconstruction for image-guided radiotherapy.

### REFERENCES

Bergner F, Berkus T, Oelhafen M, Kunz P, Pan T, Grimmer R, Ritschl L and Kachelriess M 2010 An investigation of 4D cone-beam CT algorithms for slowly rotating scanners Med. Phys. 37(9), 5044-5053.
Candes E, Romberg J and Tao T 2006 Robust uncertainty principles: Exact signal reconstruction from highly incomplete frequency information IEEE Trans. Inf. Theory 52(2), 489-509.
Chan C, Fulton R, Barnett R, Feng D D and Meikle S 2014 Postreconstruction nonlocal means fi of whole-body PET with an anatomical prior IEEE Trans. Image Process. 33(3), 636-650.
Chan C, Fulton R, Feng D D and Meikle S 2009 Regularized image reconstruction with an anatomically adaptive prior for positron emission tomography Phys. Med. Biol. 54(24), 7379-7400.
Chantas G, Galatsanos N, Molina R and Katsaggelos A 2010 Variational Bayesian Image Restoration With a Product of Spatially Weighted Total Variation Image Priors IEEE Transactions on Image Processing 19(2), 351-362.
Chen G H, Tang J and Leng S 2008 Prior image constrained compressed sensing (PICCS): A method to accurately reconstruct dynamic CT images from highly undersampled projection data sets Med. Phys. 35(2), 660-663.
Chen Q, Montesinos P, Sen Sun Q, Heng P A and Xia D S 2010 Adaptive total variation denoising based on diff curvature Image Vision. Comput. 28(3), 298-306.
Choi K, Wang J, Zhu L, Suh T S, Boyd S and Xing L 2010 Compressed sensing based cone-beam computed tomography reconstruction with a fi method Med. Phys. 37(9), 5113-5125.
Cropp R J 2011 Implementation of respiratory-correlated cone-beam CT on Varian linac systems. Master's thesis, The University Of British Columbia. pp. 48.
Dong W, Yang X and Shi G 2013 Compressive sensing via reweighted TV and nonlocal sparsity regularisation Electron. Lett. 49(3), 184-185.
Donoho D 2006 Compressed sensing IEEE Trans. Inf. Theory 52(4), 1289-1306. Feldkamp L, Davis L and Kress J 1984 Practical cone-beam algorithm J. Opt. Soc. Am. A Opt. Image. Sci. Vis. 1(6), 612-619.
Haas B, Coradi T, Scholz M, Kunz P, Huber M, Oppitz U, Andre L, Lengkeek V, Huyskens D, van Esch A and Reddick R 2008 Automatic segmentation of thoracic and pelvic CT images for radiotherapy planning using implicit anatomic knowledge and organ-specific segmentation strategies Phys. Med. Biol. 53 (6), 1751-1771.
Harsolia A, Hugo G D, Kestin L L, Grills I S and Yan D 2008 Dosimetric advantages of four-dimensional adaptive image-guided radiotherapy for lung tumors using online cone-beam computed tomography Int. J. Radiat. Oncol. 70(2), 582-589.
Jia X, Lou Y, Li R, Song W Y and Jiang S B 2010 GPU-based fast cone beam CT reconstruction from undersampled and noisy projection data via total variation Med. Phys. 37(4), 1757-1760.
Kavanagh A, Evans P M, Hansen V N and Webb S 2009 Obtaining breathing patterns from any sequential thoracic x-ray image set Phys. Med. Biol. 54(16), 4879-4888.
Lauzier P T, Tang J and Chen G H 2012 Prior image constrained compressed sensing: Implementation and performance evaluation Med. Phys. 39(1), 66-80.
Leng S, Tang J, Zambelli J, Nett B, Tolakanahalli R and Chen G H 2008 High temporal resolution and streak-free four-dimensional cone-beam computed tomography Phys. Med. Biol. 53(20), 5653-5673.
Leng S, Zambelli J, Tolakanahalli R, Nett B, Munro P, Star-Lack J, Paliwal B and Chen G H 2008 Streaking artifacts reduction in four-dimensional cone-beam computed tomography Med. Phys. 35(10), 4649-4659.
Liu Y, Ma J, Fan Y and Liang Z 2012 Adaptive-weighted total variation minimization for sparse data toward low-dose x-ray computed tomography image reconstruction Phys. Med. Biol. 57(23), 7923-7956.
Lu J, Guerrero T M, Munro P, Jeung A, Chi P C M, Baiter P, Zhu X R, Mohan R and Pan T 2007 Four-dimensional cone beam CT with adaptive gantry rotation and adaptive data sampling Med. Phys. 34(9), 3520-3529.
Mckinnon G and Bates R 1981 Towards imaging the beating heart usefully with a conventional CT scanner IEEE Trans. Biomed. Eng. 28(2), 123-127.
Niu T and Zhu L 2012 Accelerated barrier optimization compressed sensing (ABOCS) reconstruction for cone-beam CT: Phantom studies Med. Phys. 39(7), 4588-4598.
Rit S, Oliva M V, Brousmiche S, Labarbe R, Sarrut D and Sharp G C 2014 The Reconstruction Toolkit (RTK), an open-source cone-beam CT reconstruction toolkit based on the Insight Toolkit (ITK) J. Phys.: Conf. Ser. 489(1), 012079.
Ritschl L, Bergner F, Fleischmann C and Kachelriess M 2011 Improved total variation-based CT image reconstruction applied to clinical data Phys. Med. Biol. 56(6), 1545-1561.
Segars W P, Sturgeon G, Mendonca S, Grimes J and Tsui B M W 2010 4D XCAT phantom for multimodality imaging research Med. Phys. 37(9), 4902-4915.
Sidky E Y and Pan X 2008 Image reconstruction in circular cone-beam computed tomog-raphy by constrained, total-variation minimization Phys. Med. Biol. 53(17), 4777-4807.
Sonke J, Zijp L, Remeijer P and van Herk M 2005 Respiratory correlated cone beam CT Med. Phys. 32(4), 1176-1186.
Strong D, Blomgren P and Chan T 1997 Spatially adaptive local feature-driven total variation minimizing image restoration Proc. SPIE Annu. Meeting 3167, 222-233.
Tian Z, Jia X, Yuan K, Pan T and Jiang S B 2011 Low-dose CT reconstruction via edge-preserving total variation regularization Phys. Med. Biol. 56(18), 5949-5967.
van Rikxoort E M, de Hoop B, Viergever M A, Prokop M and van Ginneken B 2009 Automatic lung segmentation from thoracic computed tomography scans using a hybrid approach with error detection Med. Phys. 36(7), 2934-2947.
Vandemeulebroucke J, Bernard O, Rit S, Kybic J, Clarysse P and Sarrut D 2012 Automated segmentation of a motion mask to preserve sliding motion in deformable registration of thoracic CT Med. Phys. 39(2),1006-1015.
Volpi S, Antonelli M, Lazzerini B, Marcelloni F and Stefanescu D 2009 Segmentation and reconstruction of the lung and the mediastinum volumes in CT images ISABEL 2009 pp. 1-6.
Wang Z, Bovik A, Sheikh H and Simoncelli E 2004 Image quality assessment: From error visibility to structural similarity IEEE Trans. Image Process. 13(4), 600-612.
Yuan Q, Zhang L and Shen H 2013 Regional spatially adaptive total variation super-resolution with spatial information fi and clustering IEEE Trans. Image Process. 22(6), 2327-2342.
Zeng G and Gullberg G 2000 Unmatched projector/back projector pairs in an iterative reconstruction algorithm IEEE Trans. Med. Imag. 19(5), 548-555.
Zheng Z, Sun M, Pavkovich J and Star-Lack J 2011 Fast 4D cone-beam reconstruction using the McKinnon-Bates algorithm with truncation correction and nonlinear fi Proc. SPIE 7961, 79612U-79612U-8.

Interpretation

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, FIG., or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A method of adaptive suppression of over-smoothing in noise/artifact reduction techniques such compressed-sensing strategies for anatomy imaging modalities, the method including the steps of:
(a) inputting an imaging modality image;
(b) identifying at least one anatomical structure of interest in the imaging modality image;
(c) extracting intensity, gradient, or other image-related information of the identified anatomical structures from the imaging modality image; and
(d) adaptively suppressing over-smoothing in noise/artefact reduction at the anatomical structures of interest using the information of the anatomical structures extracted previously.

2. A method as claimed in claim 1 wherein said anatomy imaging modalities comprises one of Computed Tomography (CT) or Magnetic Resonance Imaging (MRI).

3. A method as claimed in claim 2 wherein said imaging modality comprises Cone Beam Computed Tomography (CBCT)

4. A method as claimed in any previous claim wherein said noise/artifact reduction techniques include compressed sensing.

5. A method as claimed in claim 4 wherein said compressed sensing includes total-variation minimization.

6. A method as claimed in any previous claim wherein said at least one anatomical structure of interest include at least one of a soft tissue region, lung or airway region, bony anatomy, the pulmonary region.

7. A method as claimed in any previous claim wherein said at least one anatomical structure of interest is identified by examining features which can be used to identify them in the image modality such as shapes, attenuation coefficients, sizes or positions.

8. A method as claimed in any previous claim wherein said imaging modality is applied to at least one of the head region, neck region, chest or the prostate.

9. A method of improving CBCT image reconstruction of a first body having anatomical structures, the method including the steps of:
(a) determining a segmented anatomy prior for the first body delineating the anatomical structures; and
(b) utilizing the segmented anatomy prior to modulate the reconstruction procedure of the CBCT image.

10. A method as claimed in claim 9 wherein said step (b) utilizes an iterative minimization process and further includes iteratively applying the segmented anatomy prior with the CBCT image with a reducing impact factor during successive iterations.

11. A method as claimed in claims 9 and 10 wherein said step (b) further includes iterative alternations between a projection onto convex sets and a total variation minimization.

12. A method as claimed in any previous claims 9 and 10 wherein the reconstruction procedure comprises minimization of an objective function that combines data fidelity and total variation.

13. A method as claimed in any previous claims 9 to 12 wherein said the minimization procedure includes the 11 norm of the difference between the CBCT image and a prior image.

14. A method as claimed in claim 9 wherein said first body comprises the thoracic region, and said segmented anatomy prior includes at least one of soft tissue, the lungs and airways, pulmonary details and bone anatomy.

15. A method as claimed in claim 10 wherein said impact factor is geometrically reduced between iterations.

16. An apparatus when implementing the method of any of claims 1 to 15.
